(19) United States
(12) Patent Application Publication
(10) Pub. No.: US 2014/0039585 A1
(43) Pub. Date: Feb. 6, 2014

(54) COLD THERAPY DEVICE WITH INDIVIDUAL COMPARTMENTS

(71) Applicant: Talus Research + Design, LLC, Basalt, CO (US)

(72) Inventors: J. David Stienmier, Arvada, CO (US); Eric R. Kelso, Erie, CO (US)

(73) Assignee: Talus Research + Design, LLC, Basalt, CO (US)

(21) Appl. No.: 14/032,526

(22) Filed: Sep. 20, 2013

Related U.S. Application Data
(62) Division of application No. 13/365,011, filed on Feb. 2, 2012.

(51) Int. Cl. A61F 7/10

(52) U.S. Cl. CPC A61F 7/106
USPC 607/114; 53/474

(57) ABSTRACT

A cold therapy apparatus, a cold therapy system and a method of using a cold therapy apparatus, including a method for lowering the body temperatures in a subject.

### CROSS-REFERENCE TO RELATED APPLICATIONS

This application relates to and claims the benefit and priority to U.S. Provisional Application No. 61/438,884, filed Feb. 2, 2011.

### BACKGROUND

The present developments are directed generally to cooling apparatuses for lessening physical body trauma such as bead and neck trauma typically after injury. In some examples, this may include lowering tympanic temperature. Current methods and apparatuses have some limitations such as suffering from ineffective cooling, or contrarily overcooling of the neck or head. Other limitations may include time period efficacy, as in how long and effectively an apparatus may cool the neck before warming to a point of less or non effectiveness. A cold therapy apparatus or device hereof could lower tympanic temperature effectively and result in similar outcomes to experiments performed with ice/water mixtures.

Thus, it may be found desirable to provide cold therapy devices, apparatuses, and/or methods, that apply cold to lower body temperature, such as tympanic or other body temperature in a cost-efficient, time effective and compact manner, and in some implementations providing safeguards that may effect cooling while minimising the risk of further injury to a patient through exposure to excessive localised cooling.

### SUMMARY

The present developments may be directed to cold therapy apparatuses, systems, and/or methods, but more particularly, in some implementations, to cold therapy apparatuses of the type used to cool human body temperatures to lessen injury and/or trauma. In some implementations, such an apparatus may be for application to the head and/or neck to lessen or reduce trauma to the head or neck after injury or illness, e.g., but not limbed to, after traumatic brain or spinal injury or in the early stages of acute ischemic stroke. The present disclosure thus relates to a cold therapy apparatus and/or system and/or method by which the cold therapy may be employed.

A cold therapy apparatus or device hereof may provide at least two compartments comprising a water compartment and a discrete cooling chemical mixture compartment. In such an example, the water compartment may be disposed in use adjacent to and preferably held in contact with a portion of the body which may benefit from cold therapy, e.g., the head, neck, joints, feet, hands, arms, legs, torso, ribs, stomach, elbow, ankle, wrist, shoulder, knee, buttocks, hips, pelvis, and the like. For example, the neck or head can be used (e.g., next the back, sides, or front of the neck or head, and/or e.g., adjacent to a carotid artery or jugular vein). The cooling chemical mixture can then be disposed adjacent the water compartment and may optionally use one or more of ammonium nitrate, sodium acetate trihydrate, and water. In addition, further compartments or baffles can be used within a device hereof to protect the patient's skin from direct contact with the chemical mixture compartment and thereby reduce the likelihood that the skin contact temperature will drop below 0 degrees Celsius. Outcomes may be similar to or better than outcomes in experiments performed with an ice and water mixture.

### BRIEF DESCRIPTION OF THE DRAWINGS

In the drawings:

FIG. 1 provides a front perspective view of a coin therapy apparatus as used for optional application to the neck of a subject;

FIG. 2 provides a rear perspective view of a cold therapy apparatus like that shown in FIG. 1;

FIG. 3 provides a rear perspective view of a cold therapy apparatus like that shown in FIG. 1, though not shown in use;

FIG. 4 provides a cross section of side view of a cold therapy apparatus like that shown in FIG. 3;

FIG. 5 provides a cross section of side view of a cold therapy apparatus, showing optional application to the neck of a subject;

FIG. 6 which includes sub-part FIGS. 6a and 6b, provides cross sectional views from a top view of respective cold therapy apparatuses hereof;

FIG. 7 provides a schematic of a principle of operation of a cold therapy apparatus according hereto;

FIG. 8 provides a graphical presentation of data relating to experimental set up 1 for transferring heat and maintaining the chemical mixture and water temperatures using a cold therapy apparatus of the present developments.

FIG. 9 provides a graphical presentation of data relating to experimental set up 2 for transferring heat and maintaining the chemical mixture and wafer temperatures using a cold therapy apparatus of the present developments.

FIG. 10 provides a graphical presentation of data relating to experimental set up 3 for transferring heat and maintaining the chemical mixture and water temperatures using a cold therapy apparatus of the present developments.

FIG. 11 provides a graphical presentation of data relating temperature to time for a number of subjects using a cold therapy apparatus of the present developments.

FIG. 12 provides a graphical presentation of data relating to temperature to time for a number of subjects using a cold therapy apparatus of the present developments.

FIG. 13 provides a graphical presentation of data relating temperature to time for a number of subjects using a cold therapy apparatus of the present developments.

FIG. 14 provides a graphical presentation of data relating temperature to time for a number of subjects using a cold therapy apparatus of the present developments.

FIG. 15 provides a flow chart of how a cold therapy apparatus may be used on a subject by activation and application.

### DETAILED DESCRIPTION

As discussed in further detail below, experimental results show that ice applied to the body can provide therapeutic benefit, as for example as applied to a neck which may thus provide an effective means for lowering, a sustained and measurable amount, body temperature, such as or as evidenced by tympanic temperature. The result, measured as drop in temperature from, a baseline level, can be improved by applying ice to the head in addition to the neck. Contributing factors to tympanic temperature drop are believed to be heat conduction through neck and head, resulting in a cooling of the carotid and tissue surrounding the ear. In addition, counter-current heat exchange occurs between the carotid and jugular vein. This effect, may he increased when one or more regions of the head are iced in addition to the neck.

The developments hereof relate to a cold therapy apparatus or system typically configured to achieve one or more of easy and rapid transportation, use, application, and/or removal, and a method of use thereof. In many implementations, the cold therapy apparatus may have a water barrier and a chemical cold pack contained within a single structure, though in separate but adjacent compartments, adapted for use in a cold therapy apparatus to be applied to a subject and/or patient.

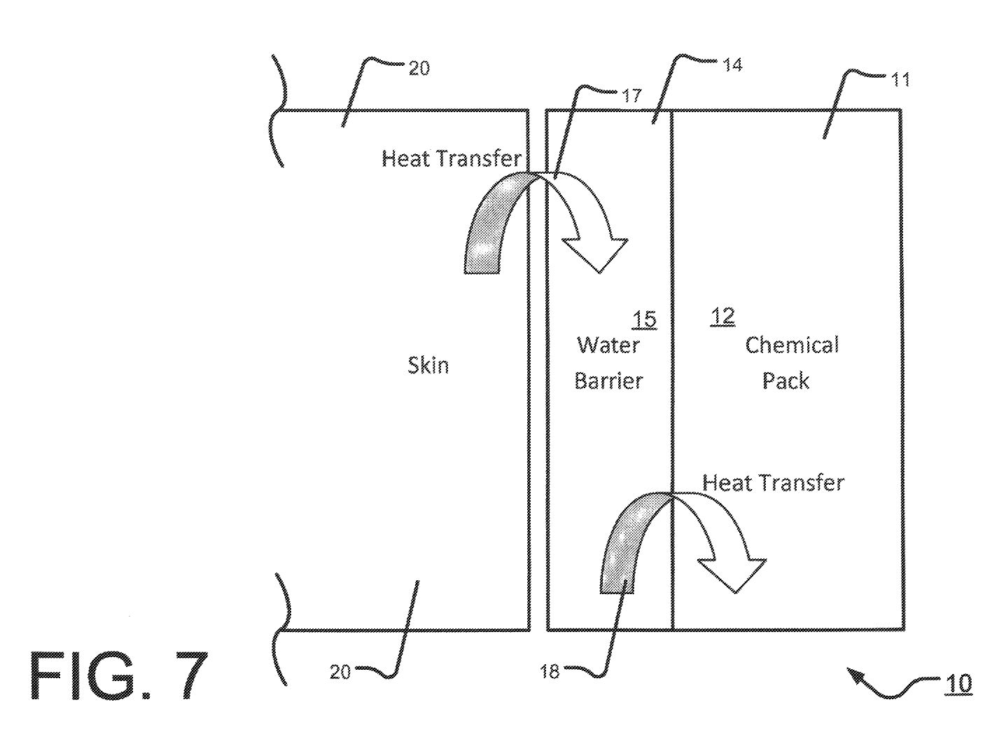

FIG. 7, as further described below, provides a schematic for a cold therapy apparatus showing a water compartment disposed between a subject's skin and a cooling chemical pack compartment in use for maintaining a more controlled skin and/or adjacent vascular temperature, as for example, for reducing the neck tympanic temperature by removing heat by use of the cold therapy apparatus.

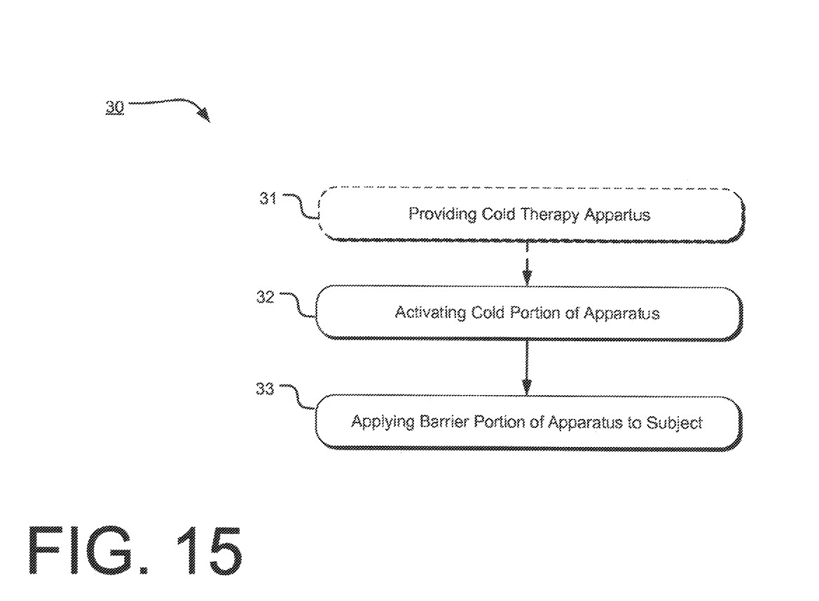

FIG. 15 shows a flow diagram 30 of how a cold therapy apparatus can be activated in operation 32, and applied in operation 33, for use on a subject to provide cold therapy. The optional provision operation 31 may also/alternatively be included, whether my manufacture or other supply and/or provision operation.

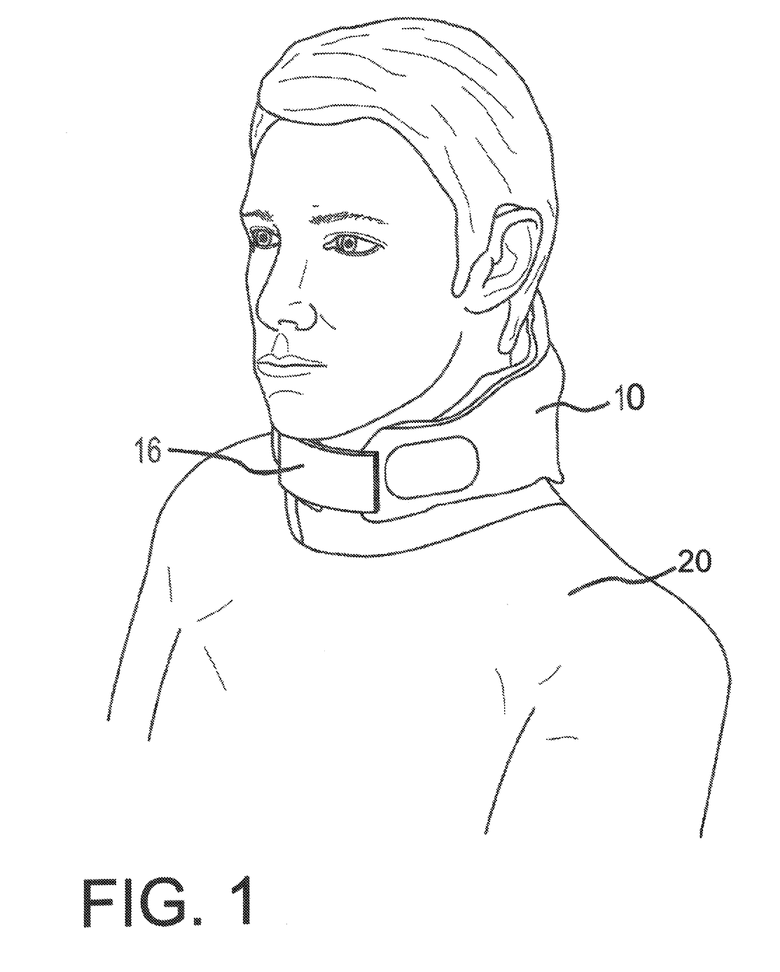

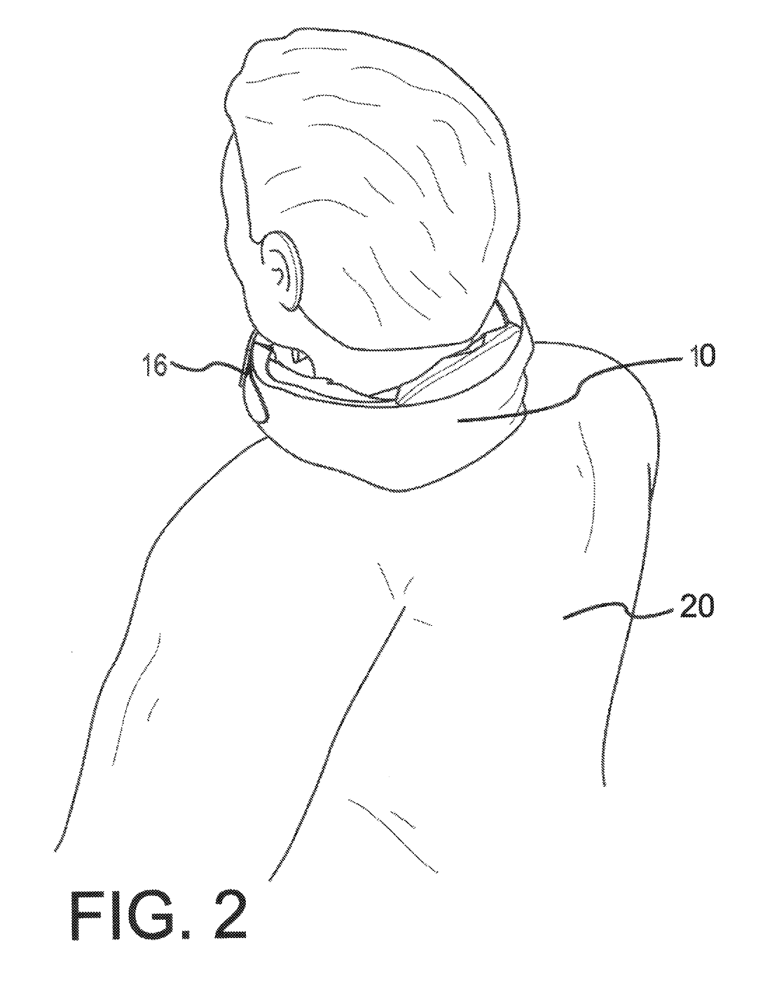

FIGS. 1-6 provide drawing figures of a cold therapy apparatus 10 of some implementations of the present developments. In FIGS. 1 and 2, the apparatus or device 10 is shown connected to a subject or patient 20. A fastening device or system 16 is also introduced.

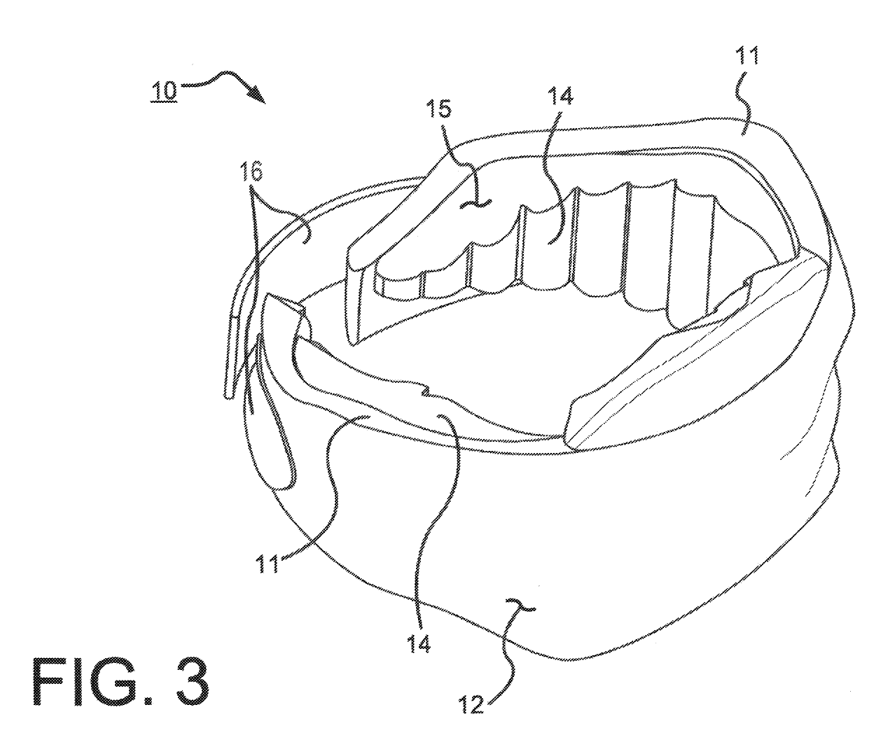

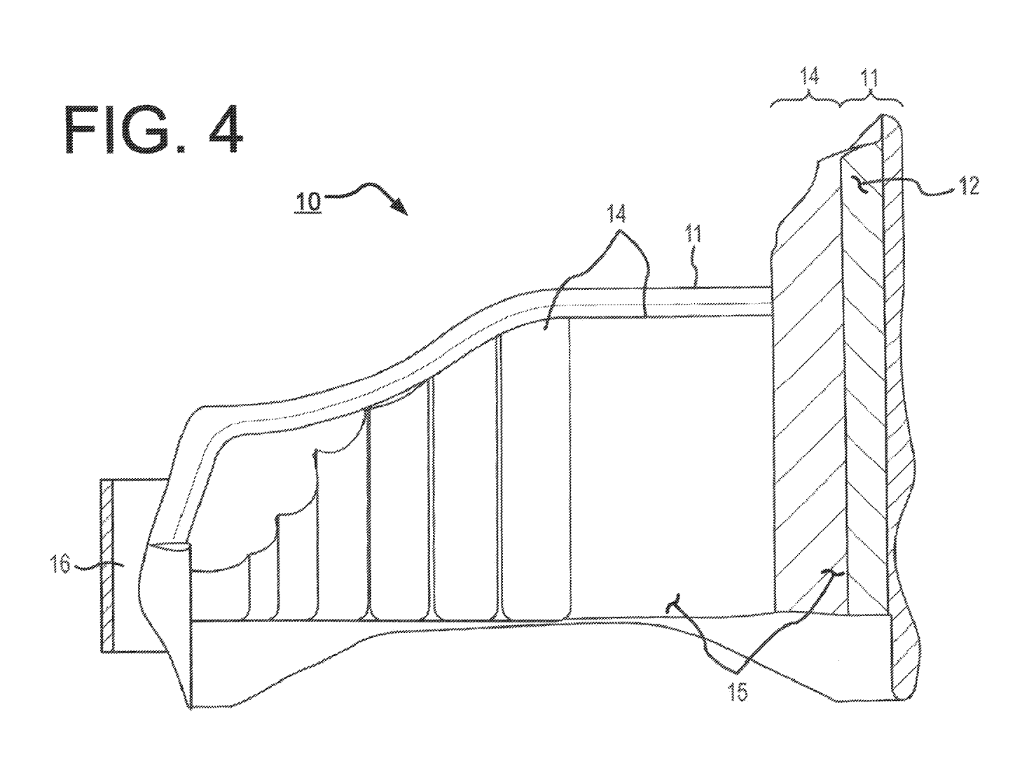

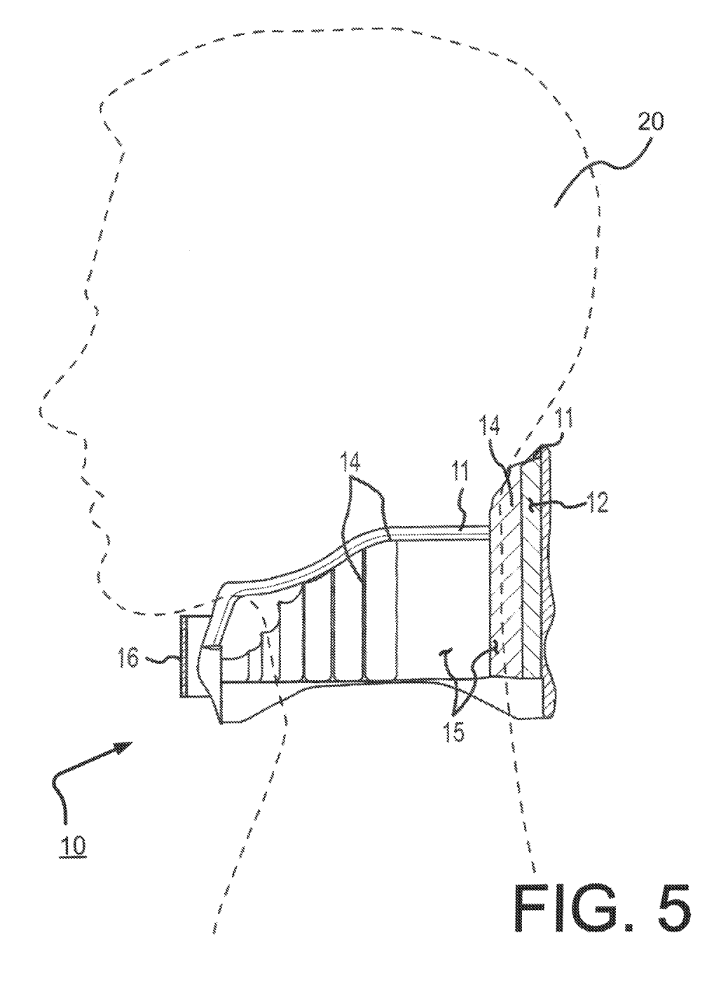

FIGS. 3 and 4 provide a little more detail of the device 10. A chemical pack compartment 11, having disposed therein a chemical mixture 12, is provided for cooling of the neck of a subject 20. A water compartment 14, having water 15 disposed therein, is disposed adjacent the chemical compartment 11. In use, the water compartment 14 is positioned adjacent and/or against the skin of the patient 20, see e.g., the cross-section of FIG. 5, and is disposed between the skin and the chemical compartment 11. The water 15 in the water compartment 14 transfers heat from the subject 20 into the water 15, and from there transfers heat to the chemical mixture 12 in the chemical pack compartment 11. These first and second heat transfer movements are shown by the schematic arrows 17 and 18 in FIG. 7.

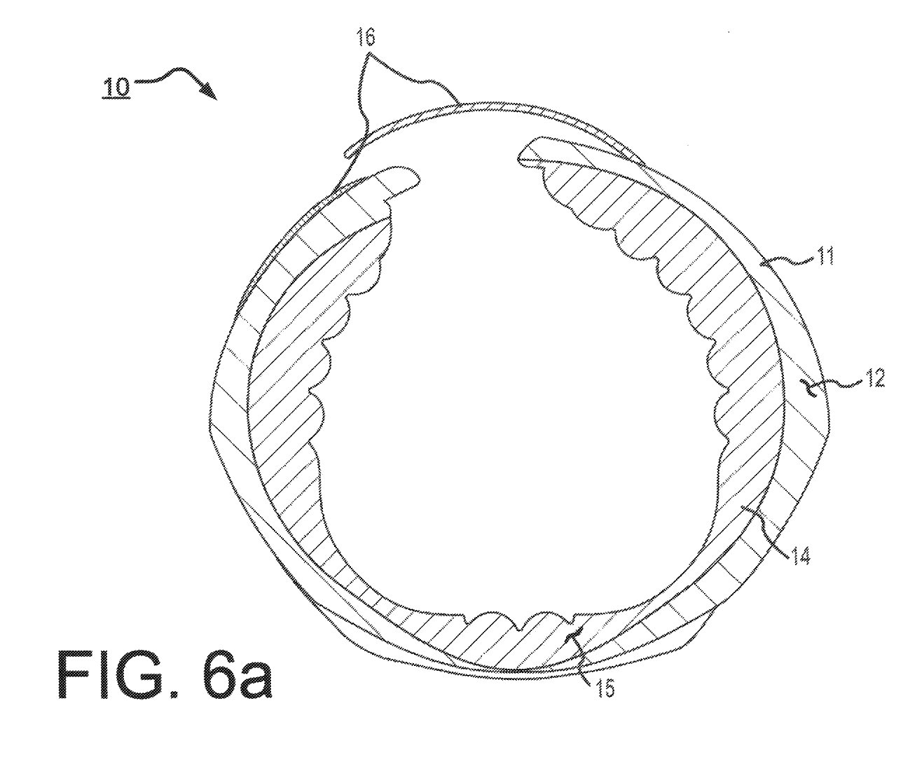

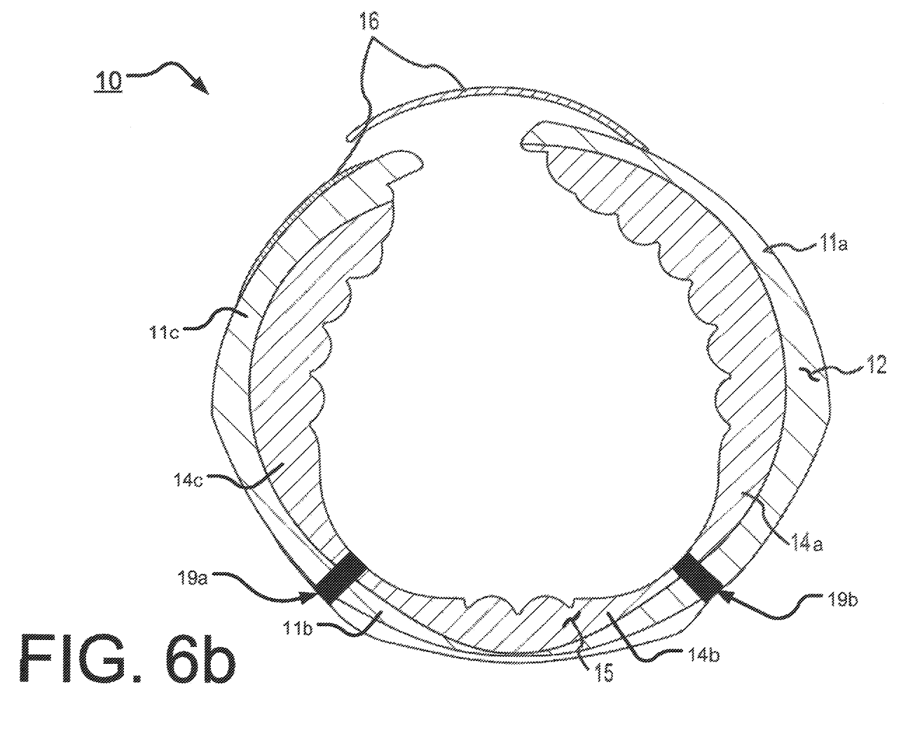

The respective compartments, e.g., 11 and 14, can be continuous or separated into 1, 2, 3, 4, 5 or more compartments, for each of the chemical or water compartments. FIG. 6, in sub-part FIGS. 6a and 6b, also shows schematically the respective compartments 11 and 14 and the filling materials 12 and 15. In FIG. 6a, these are shown as relative continuous compartments around the substantial part of the circumference of the device 10. In FIG. 6b, these are shown as separated by barriers 19a and 19b into three sub-part compartments, chemical mixture compartments 11a, 11b, 11c and water compartments 14a, 14b, 14c. These are schematic representations of an alternative implementation and will be discussed again below in reference to data generated in reference to Table 1, below. Small side pouch in Table 1 and accompanying description refers also to compartments 11c and 14c; large side pouch in Table 1 and accompanying description refers also to compartments 11a sand 14a; and, center pouch to compartments 11b and 14b.

An attachment device 16 can also be provided on a cold therapy apparatus 10 to attach the apparatus 10 onto a subject 20. The attachment device 16 can include any suitable attaching mechanism, e.g., but not limited to a clasp, a button, a zipper, VELCRO™ or like hook and loop fasteners, a tie, or the like or alternative connection devices or means.

As shown in FIG. 7 (schematic layout for a device that cools the skin using a chemical pack layer and water barrier), there may be one or several design constraints considered. First, the water barrier next to the subject's skin will preferably freeze completely, or at least not completely within about 10-60 minutes, such as a preferable time period of about 25-35 minutes, or in some implementations about 30 minutes; e.g., times may be: 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, or 60 minutes, or any range or value therein or even reasonably outside this range, including if the device is activated at 0 Deg C. Second, if activated at warmer temperatures (e.g., 5, 10, 15, 20, 25, or 28 Deg C.) the water layer must reach a sufficiently low temperature in a relatively short period time to provide effective cooling. Also, the volume for construction of a device that meets the above criteria should be reasonable for the application.

Cooling or endothermic chemical mixtures (e.g., as known in the art or described herein) can be provided as associated with compartment 11. The cooling or endothermic mixture can be provided as separated or mixed with the water component of the chemical mixture 12. The separated mixture can be provided in mixable components or containers, such as activated or that can be activated, e.g., but not limited to, breakable, operable, releasable, or mixable chemical mixture containing containers, packaging, packets, mechanisms, or the like, to mechanically, physically, or chemically add the chemical mixture to the water to provide the cooling or endothermic chemical mixture further comprising water as chemical mixture 12. The cooling or chemical mixture can comprise any suitable cooling or endothermic chemical that can be activated when mixed with water. Non-limiting examples can include one or more of urea, ammonium nitrate, sodium acetate, sodium hydrate, and/or sodium acetate trihydrate. Such components can be provided in various forms and/or mechanisms to provide chemical mixture 12 for compartment 11. Typically, this may include a device or container within compartment 11, compartment 11 otherwise filled with water or the like. Then, such a device or mechanism may hold the chemicals, e.g., in solid or liquid form; typically in solid salts form; and, this may device or mechanism may be activated as by rupturing or breaking open the device or mechanism to release the chemical salts into the water with in compartment 11, mix and/or react with the water and thereby create a cold mixture within the chemically-driven cold compartment. The amounts of salts relative to the water may be chosen to provide the appropriate level of temperature relative to the water barrier 14 to provide desirable heat transfer therefrom and from the patient as well (see FIG. 7).

The present developments also provide in some implementations, a method comprising: activating the cold therapy apparatus by causing the chemical mixture to interact with water provided in the chemical pack compartment; and applying the activated cold therapy apparatus to subject within an application time period after trauma (e.g., within minutes in preferred implementations, or perhaps within longer periods, 10, 15, 20, 30, 60 or even more time in some other implementations), injury or stroke to provide cold therapy to the subject for a cooling time period of at least 25-35 minutes; again, other periods, shorter or longer, may be desired and effected as well. FIG. 15 shows a flow diagram 30 of how a cold therapy apparatus can be activated 32, and applied 33, for use on a subject to provide cold therapy. As also shown in FIG. 15, also provided may be a preliminary optional operation 31 of providing a cold therapy apparatus, comprising: a chemical pack compartment comprising a chemical mixture for cooling a portion of the body of a subject; a water compartment displaced between the subject and the internal chemical pack compartment for transferring heat from the subject to the chemical pack; and an attachment device for securing the apparatus to a subject such that the water compartment is adjacent to the body of the subject. In some examples, the body part may be the head and/or neck of a subject; however other parts of the body may also/alternatively be used. Other parts of the head may include the forehead, top or sides of the head; or other body parts such as the neck and shoulder area, clavicle area, or shoulders, knees, elbows, ankles, or any other body part which might benefit from application of cooling for therapy.

Results of some examples of such mixtures relative to water to determine effectiveness of cooling, particularly as these may approach cooling of an ice water mixture directly on the skin are set forth below. The results indicate that a chemical pack and water barrier that is approximately 1.5 inches thick, e.g., about or substantially 1-2, 1.2-1.8, 1.3-1.7, 1.4-1.6, e.g., 1.0. 1.1, 1.2, 1.3, 1.4, 1.5, 1.6, 1.7, 1.8, 1.9, 2.0, inches, or any range or value therein, is suitable to provide the desired results according to the present developments. Additionally a chemical mixture used for cooling the adjacent water compartment maintains the water temperature close to freezing (e.g., but not limited to −1, −0.5, −0.4, −0.3, −0.2, −0.1, −0.2, −0.3, 0.4, 0.5, 0.6, 0.7, 0.8, 0.9. 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, degrees Celsius, or any range or value therein).

A baseline value for understanding how much heat can be removed from the water barrier is established by determining how much ice (starting at 0 Deg C.) direct skin contact can melt over a 30 minute period. Suitable volumes include 100-500 ml of the water and/or chemical mixture compartments and/or volumes. As a non-limiting example, a volume of 325 ml of liquid water is frozen and placed in contact with 25 square inches of skin on the neck. After a period of 30 minutes, 120 ml of water is melted. This number provides a baseline for which to design the remaining experiments around. Calculations below show the amount of heat removed item the neck during this particular non-limiting test:

Q=ΔHfus*W=333.55 J/g*120 g=40 kJ

where Q=Heat Removed (Joules), ΔHfus=Enthalpy of Fusion for Water (Joules/Gram), and W=Weight of Ice Melted (Grams).

The 40 kJ removed from the neck represents the maximum amount of heat that can be removed given it is unlikely the barrier will remain at 0 degrees Celsius during the entire time of application.

Note that the amount of energy required to freeze a gram of water is equivalent to the amount of energy needed to drop one gram of water from 79.8 Deg C. to 0 Deg C.

ΔHfus/C=(333.55 J/g)/(4.1813 J/g*K)=79.8 Celsius,

where ΔHfus=Enthalpy of Fusion for Water (Joules/Gram) and C=Specific Heat of Water.

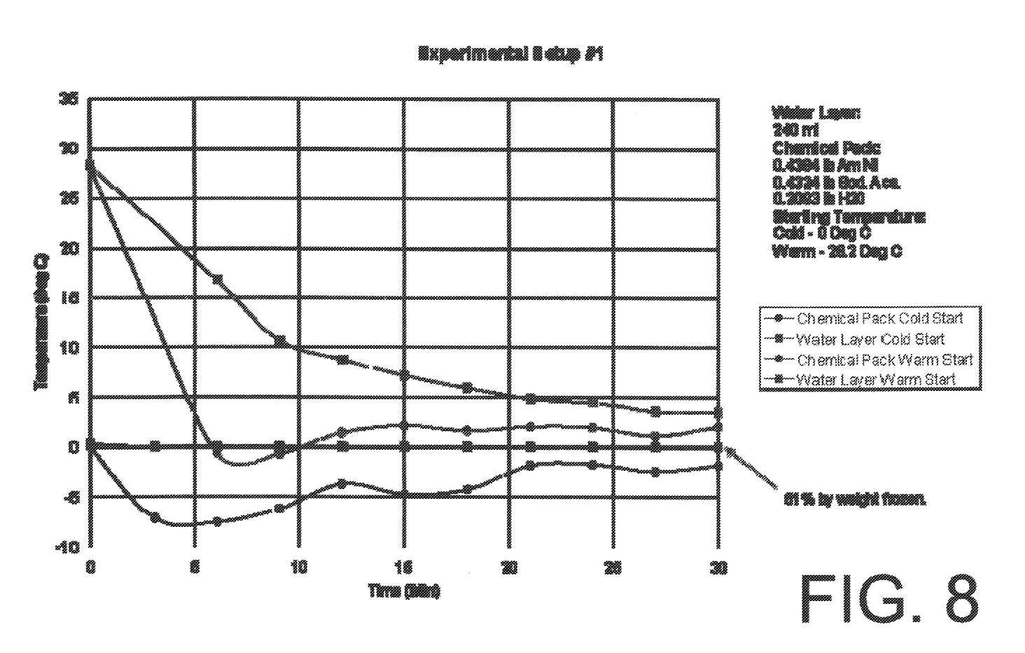

In the experiments below, see particularly FIGS. 8-10, the starting temperature for warm water is 28 C. The energy needed to cool off one gram of water from 28 C. to 0 C is 117 J. Therefore, if the device starts at 28 deg C. and the water layer is completely frozen after 30 minutes, about 25% of the transferred energy will go to cooling the water layer and 75% will go to freezing the water.

Understanding the dynamics above allows for an iterative design process in which the amount of water and the amount of cooling or endothermic chemical mixture in compartment 11 can be provided for optimum performance, as well as the amount of water in the barrier layer (compartment 14), which alone or together can be designed to allow for maximum cooling, cooling in the shortest period, cooling for the longest duration, and other possible scenarios. There may be a design tradeoff that occurs as the water barrier volume is decreased. By decreasing the volume and thickness of the water barrier the cooling effect on the skin will be achieved more quickly. However, it must be considered that a thinner water barrier provides less protection from sub-zero temperatures and results in cooling for a shorter period of time. In the experimental setups below, a water barrier (representative of compartment 14) and a chemical pack (representative of compartment 11) were placed in contact with one another in an adiabatic environment (heat transfer is substantially only allowed to occur between the chemical pack and the water layer). For the following three sets of results, FIGS. 8, 9 and 10, twenty-five (25) square inches of contact between discrete compartments of a water barrier layer and chemical mixture layer were used. Varied chemical concentrations and varied thicknesses of water barrier layer were used. Temperature was measured as a function of time in both the chemical mixture compartment and in the water barrier compartment; each for a period of 30 minutes after activating the chemical mixture.

As generally shown in FIG. 8, Graph of temperature vs. time for experimental setup 1, the water layer was selected to be 240 ml to ensure the water layer would not freeze completely if the starting temperature was 0 Deg C. Starting at 0 Deg C., over a period of 30 minutes 51% of the water barrier layer froze. When the apparatus was started at 28 Deg C., it took nearly twenty minutes to reduce the water barrier to temperatures below 5 deg C. The chemical pack temperature remained below freezing for the entire experiment. The data in FIG. 8 leads to the conclusion that a thinner water barrier and less water in the chemical pack will lead to more effective cooling of the water barrier with little possibility of dropping the water barrier below 0 Deg C. within 30 minutes of activation.

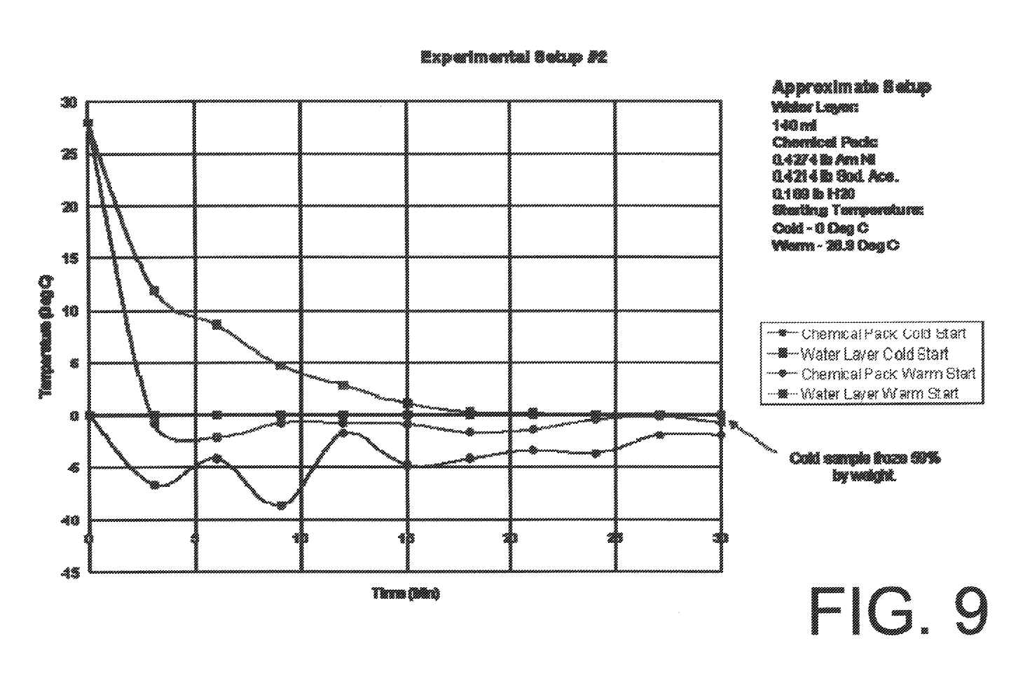

As generally shown in FIG. 9, Graph of temperature vs. time for experimented setup 2, the amount of water in the chemical pack was reduced as well as the amount of water in the barrier layer. For the case where the apparatus was started close to 0 Deg C., 59% of the water layer froze, leaving a low likelihood the water layer will drop below 0 Deg C. within 30 minutes of activation. In the case where the apparatus was started at 29 Deg C., there were faster drops in temperature for the water barrier than in experimental setup 1, FIG 8. Temperature of the water barrier dropped below 5 Deg C. within 10 minutes of activation.

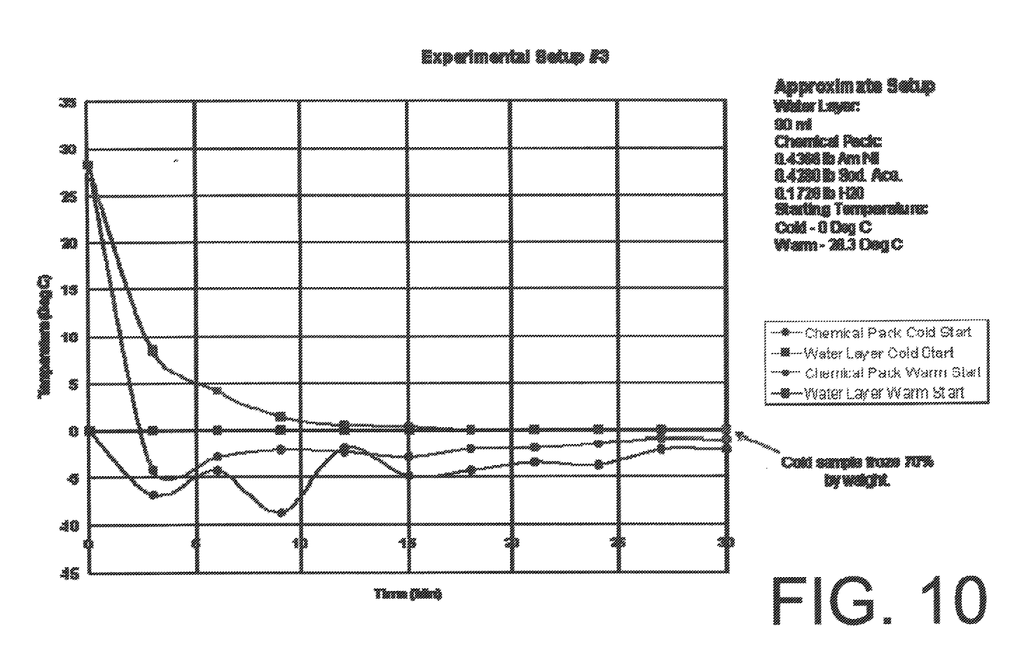

As generally shown in FIG. 10, Graph of temperature vs. time for experimental setup 3, the amount of water in the water in the barrier layer was reduced from that in setup 2, FIG. 9. For the case where the apparatus was started close to 0 Deg C., 70% of the water barrier froze, leaving a low likelihood that the water layer will drop below 0 Deg C. within 30 minutes of activation. In the case where the apparatus was started at 28 Deg C., there were faster drops in temperature for the water layer than in experimental setup 2, FIG 10. Temperature of the water barrier dropped below 5 Deg C. within 5 minutes of activation.

It would appear to be possible to continue this iterative process until nearly all of the water barrier freezes when the device temperature starts at 0 Deg C. Based on the results shown in experimental setups 1 through 3, FIGS. 8-10, it is reasonable to determine that a water layer that freezes almost completely when starting at 0 deg C. will also have sufficient cooling under the circumstance where the device is activated at 28 Deg C.

The chemical pack and water barrier design shown in experimental setup 3 allow for a 1.5 inch thick device assuming contact area of 25 square inches between layers of the device. This number was found by measuring the density of each chemical and calculating the necessary volume. Calculations for such a non-limiting example are summarized below;

### Thickness Calculations:

Weight of Chemicals and Water Barrier:
Ammonium Nitrate—0.4382 lb
Sodium Acetate Trihydrate—0.4473 lb
Water (Cham Pack)—0.21 lb
Water (Barrier)—0.17 lb

Volume of Chemicals and Water Barrier:
Ammonium Nitrate—197.00 cm̂3
Sodium Acetate Trihydrate—275.81 cm̂3
Water (Chem Pack)—93.35 cm̂3
Water (Barrier)—79.20 cm̂3

Densities:
Ammonium Nitrate (Pellet Form)—0.00222 lb/cm̂3
Sodium Acetate (Powder Forms)—0.00162 lb/cm̂3
Water—1 g/cm̂3=0.0020 lb/cm̂3

Total Volume:
39.38 in̂3 (cubic inches)

Thickness:
Volume/Area=25 in ̂⅗ in̂2=1.58 inches

A cold therapy device of the present developments effectively lowers body temperature and results in similar outcomes to experiments performed with ice/water mixtures. A cold therapy device of the present developments applied to the neck and/or head is an effective way to lower tympanic temperature a sustained and measurable amount. Non-limiting examples may include the use of a chemical mixture of ammonium nitrate, sodium acetate trihydrate, and water. In addition, one or more water compartments are provided to protect the subject's skin from direct or near contact with the chemical mixture compartment and reduce the likelihood that the skin contact temperature will drop below 0 degrees Celsius.

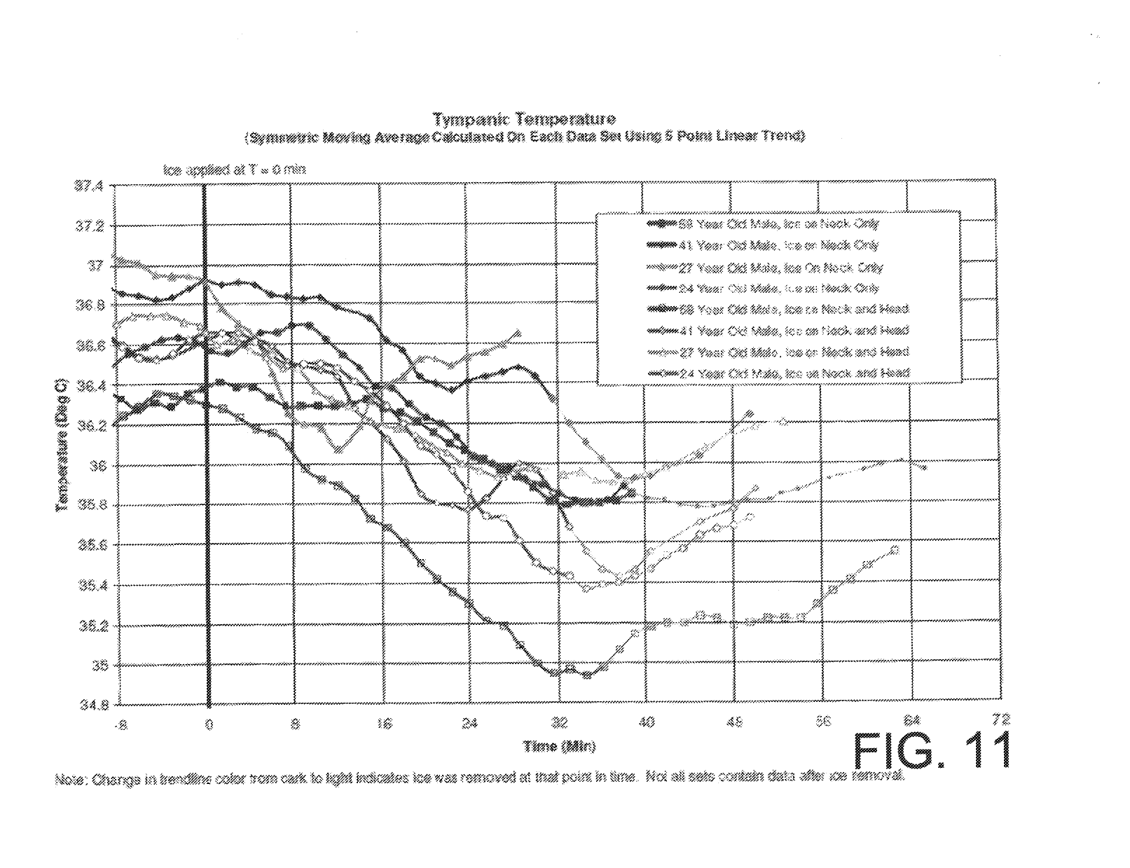

In some further examples; subjects were measured for a baseline temperature and a cold therapy device of the present developments applied to the neck, back of the head or forehead to promote cooling, such as tympanic vasculature cooling, which can include counter current heat exchange between the carotid artery and jugular vein. Ice was left on all subjects for at least 28 minutes, with data collection during and after removal of a cold therapy device of the present developments. All data was analyzed using a five-point linear fit, moving average, in an effort to display trends. The data was plotted as a temperature change, and data points are shifted down by the average of the five measurements prior to application of the ice. See FIGS. 11-13.

The average maximum drop in temperature while icing the neck and then the neck and head was 0.8 degrees C. and 1.13 degrees C. respectively. These values were calculated by first establishing a baseline temperature for each trial, calculated as the average of the five temperature measurements prior to application of the ice. The maximum drop was calculated as the difference between the baseline temperature and the lowest temperature point in the moving average data set. The maximum drop for each of the subjects was then averaged for that experimental setup. In some cases, the lowest temperature measurement was after removal of the ice. The average drop in temperature after 28.5 minutes of icing was 0.43 degrees C. while icing just the neck and 0.87 degrees C. while icing both the neck and head. These values were calculated by averaging all of the subjects' temperature measurements at one and half minute intervals for each experimental setup. The results suggest that icing the forehead and back of head, in addition to the neck, results in a measureable amount of additional cooling. Alternative implementations of one or more devices hereof may thus include forehead in addition or in alternative to carotid and/or neck and/or to target neck alone or the carotid alone or to target the head in addition to the carotid, or in other possible combinations.

As shown in FIG. 11 Tympanic Temperature vs. Time, the Figure displays the data collected across four subjects for both experimental setups. The graph displays a 5 point symmetric moving average calculated using a linear trend. Time in minutes is displayed on the x-axis and the temperature in degrees Celsius is displayed on the y-axis. Solid data points show data sets collected with ice on both sides of the neck. Hollow data points indicate experimental setups that had ice on the neck, back of head, and forehead. Lighter color lines show where ice was removed from the subject. Partway through the experiments it was determined it may be useful to continue the collection of data after ice is removed from the subject. For this reason, only certain sets contain data after the removal of ice.

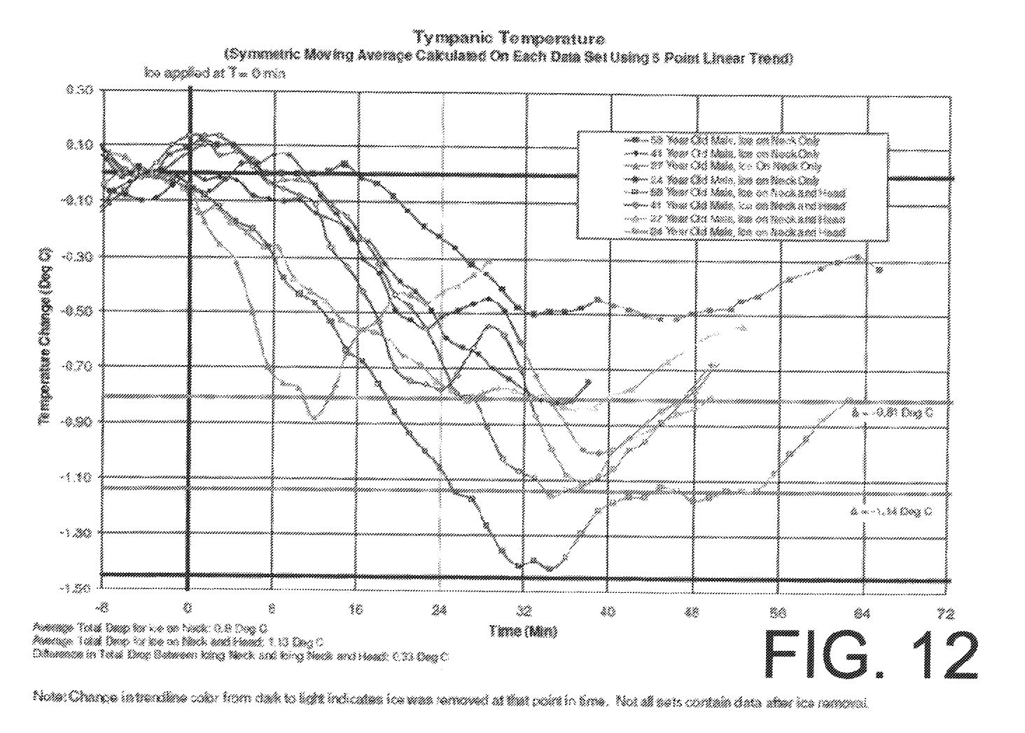

As shown in FIG. 12, Tympanic Temperature Change vs. Time, each data set from FIG. 11 is shifted by the average of the five measurements prior to application of the ice. This allows for comparison of the temperature change from each subject's baseline temperature. The maximum drop for each subject is calculated and then averaged across subjects for each experimental setup. In the experimental setup where just the neck is iced, an average maximum drop of 0.81 degrees C. is calculated. In the setup where the neck, back of head, and forehead is iced, an average maximum drop of 1.14 degrees C. is calculated. In most cases the tympanic temperature continues to stay low for a period of time after the removal of ice. This likely indicates that the cooling goes beyond the superficial skin temperature.

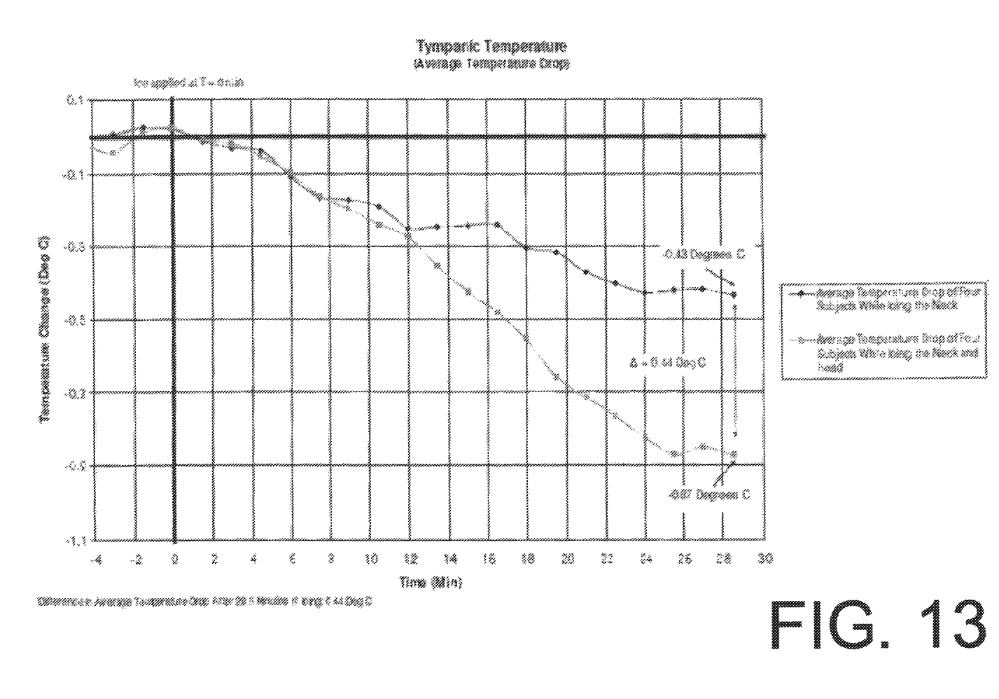

As shown in FIG. 13, Average Tympanic Temperature Change vs. Time, the average change in temperature over time for all four trials in each experimental setup is shown. The average drop in temperature is less than the maximum drop shown in FIG. 12. An additional average drop of 0.44 degrees Celsius is calculated for the subjects where ice was placed on the back of neck and forehead in addition to the neck.

Tests were also conducted to determine whether or not an ice pack composed of Ammonium Nitrate, Sodium Acetate, and Water, along with a water barrier on a subject's skin, can safely provide effective cooling that approaches the cooling scenario of an ice water mixture directly on the skin. Other known cooling components or chemicals can also be used according to the present developments.

A cold therapy mockup prior to assembly along with premeasured amounts of ammonium chloride, sodium acetate trihydrate, and water were also tested. The mockup was divided into three isolated compartments called the “small side pouch”, “center pouch”, and “large side pouch”. These were not unlike that shown in FIG. 6b, small side pouches 11c/14c, center pouches 11b/14b and large side pouches 11a/14a.

TABLE 1
Mockup Chemical Amounts

| | Ammonium | Sodium Acetate | Water |
|---|---|---|---|
| | Nitrate (lbs) | Trihydrate (lbs) | (ml) |
| Small Side Pouch | 0.12 | 0.12 | 27.2 |
| Center Pouch | 0.5 | 0.5 | 122.5 |
| Large Side Pouch | 0.27 | 0.27 | 61.2 |

Table 1 displays the amount of chemical in this non-limiting example for each compartment of the cold therapy device. Note that the chemical amounts may be partially restricted by the size and shape of the device.

As in previous trials, tympanic temperature was measured at one and half minute intervals throughout the experiment. The subject for the experiment was the same as the 24 year old male used for the ice and water testing. The subject lies on the floor for a period of ten minutes while a baseline temperature is established. The mockup chemicals were activated by injecting water into the compartments containing the ammonium nitrate and sodium acetate trihydrate. The starting temperature of the water for both the baffles and the chemical pouch is 10 degrees C. The mockup was then placed on the subject for a period of forty minutes.

The data was filtered using a five-point, linear fit, moving average to be consistent with the previous trials. The drop in temperature from placing the mockup on the subject's neck was similar to the data collected from previous experiments using ice and water.

### Results

The drop in temperature from placing the mockup on the subject's neck is similar to the data collected from previous experiments using ice and water.

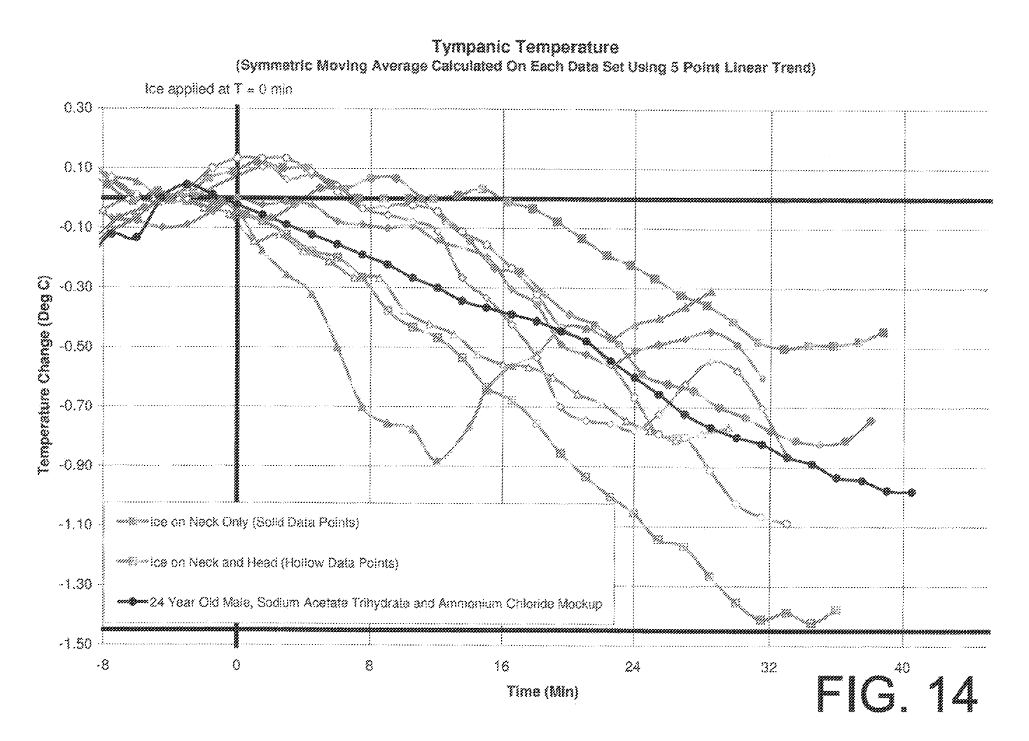

In FIG. 14, Tympanic Temperature Change vs. Time, the data is shifted down by the average of the five measurements prior to the application of the mockup. The light gray lines are experimental data collected using ice and water in previous trials. Over the forty minute period where the activated mockup is on the subject's neck there is a total drop in temperature of 0.98 degrees Celsius. These data suggest that the mockup cools the subject in a manner similar or better to the ice and water mixtures previously tested, e.g., without freezing the skin of the subject.

An apparatus such as any of the cold therapy apparatuses as described above may thus provide convenient and safe ways to effect cold therapy in such a manner as to make it highly advantageous to the operator. Ease and/or quickness of assembly and/or the ready availability of the materials to be used for the connective structures can be attractive features to an operator desiring a cost-efficient and space-efficient means of obtaining a cold therapy apparatus, system and/or method. Thus, it would not be necessary to carry any expensive, specialized cooling apparatus. Markets for use hereof may include places where a cold therapy apparatus is frequently used, such as sporting events or at the scene of a medical emergency such as a car accident. Then, easy use of the apparatus as described above may be achieved.

Apparatuses hereof may be made by any of a variety of methods and/or of a variety of materials. Shapes and sizes are not limited to those shown and described here either, as sizes and shapes may be selected to adapt to any of many alternative structures.

Although the present developments have been described with reference to preferred implementations, workers skilled in the art will recognize that changes may be made in form and detail without departing from the spirit and scope of the development described herein.

1-37. (canceled)

38. A cold therapy apparatus for cooling a subject, the cold therapy apparatus comprising a plurality of individual compartments, each of the plurality of individual compartments containing at least two components that cause an endothermic reaction when mixed together, at least one of the components being in liquid form and separated from the at least one other component until activated, each individual compartment being individually activatable to activate the endothermic reaction.

39. The cold therapy apparatus of claim 38 wherein one of the at least two components is water.

40. The cold therapy apparatus of claim 38 wherein the at least two components are separated from each other by a frangible membrane.

41. The cold therapy apparatus of claim 38 wherein there are three of the individual compartments.

42. The cold therapy apparatus of claim 38 wherein a barrier is present intermediate each of the plurality of individual compartments.

43. The cold therapy apparatus of claim 38 further including an attachment device for securing the apparatus to a subject.

44. A cold therapy apparatus for cooling a subject, the cold therapy apparatus comprising
a plurality of individual compartments, each of the plurality of individual compartments containing at least two components that cause an endothermic reaction when mixed together, at least one of the components being in liquid form and separated from the at least one other component until activated, each individual compartment being individually activatable to activate the endothermic reaction; and
a water compartment disposed between a subject and the plurality of individual compartments for transferring heat from a subject to the plurality of individual compartments.

45. The cold therapy apparatus of claim 44 wherein the water compartment is comprised of a plurality of individual sub-compartments, each containing water.

46. The cold therapy apparatus of claim 45 wherein the plurality of individual compartments and sub-compartments containing water are equal in number.

47. The cold therapy apparatus of claim 46 wherein there are three of the individual compartments and three sub-compartments containing water.

48. The cold therapy device of claim 45 further including an attachment device for securing the apparatus to a subject such that the water compartment is adjacent a subject.

49. A method for manufacturing a cold therapy apparatus for cooling a subject, comprising the steps of:
providing at least two components that cause an endothermic reaction when mixed together; and
packaging the at least two components within a common enclosure having a plurality of individual compartments each containing the at least two components.

50. The method of claim 49 wherein the step of packaging the at least two components includes separating the at least two components by means of a frangible membrane.

51. The method of claim 49 further including the step of providing a water compartment located between a subject and the plurality of individual compartments.

52. The method of claim 51 wherein the step of providing a water compartment comprises providing a water compartment having a plurality of individual water sub-compartments.
